(19) United States
(12) Patent Application Publication
(10) Pub. No.: US 2025/0255580 A1
(43) Pub. Date: Aug. 14, 2025

(54) MODULAR ULTRASONIC DEVICE, ULTRASONIC SYSTEM, AND METHOD FOR MODIFYING A MODULAR ULTRASONIC DEVICE

(71) Applicant: FRAUNHOFER-GESELLSCHAFT ZUR FÖRDERUNG DER ANGEWANDTEN FORSCHUNG E.V., Munich (DE)

(72) Inventors: Holger Hewener, Sulzbach (DE); Steffen Tretbar, Sulzbach (DE)

(21) Appl. No.: 18/867,092

(22) PCT Filed: May 19, 2022
(86) PCT No.: PCT/EP2022/063541

§ 371 Date: Nov. 19, 2024

(51) Int. Cl. A61B 8/00

(52) U.S. Cl. CPC A61B 8/4411; A61B 8/4461; A61B 8/4483; A61B 8/56

(57) ABSTRACT

A modular ultrasonic device is proposed. The modular ultrasonic device comprises a housing and at least one ultrasonic transducer. The at least one ultrasonic transducer is configured to generate and emit ultrasonic waves based on a respective control signal and to generate a respective measurement signal depending on received ultrasonic waves. The modular ultrasonic device further comprises a plurality of boards arranged in the housing, which are separably connected to one another via respective plug connectors. The plurality of boards comprises a first board with a power supply circuit configured to generate a respective power supply signal for the at least one ultrasonic transducer and the further boards of the plurality of boards. The plurality of boards further comprises at least one transmit circuit configured to generate the respective control signals for the at least one ultrasonic transducer, and a receive circuit configured to process the respective measurement signals of the at least one ultrasonic transducer. The transmit circuit and the receive circuit are either both formed on a second board of the plurality of boards or the transmit circuit is formed on the second board and the receive circuit is formed on a third board of the plurality of boards. The at least one ultrasonic transducer is formed on a fourth board of the plurality of boards or is separably connected to one of the plurality of boards.

### TECHNICAL FIELD

The present disclosure relates to the application of ultrasound. In particular, embodiments relate to a modular ultrasonic device, an ultrasonic system, and a method for modifying a modular ultrasonic device.

### BACKGROUND

Ultrasound is an established, cost-effective, real-time capable, non-invasive and universally applicable method used in many fields of medicine, but also in industrial testing and measurement technology. For the very wide range of all applications possible with ultrasound, various systems of different degrees of integration and levels of complexity are available. However, all systems on the market are dedicated, closed developments which are tailored to only a few applications and which carry their “specific intelligence” exclusively in a circuit logic embedded in the hardware or are correspondingly restricted by the hardware. This circumstance makes all these systems inflexible, causes high development costs for new applications (requirements) and a relatively long “time-to-market” in the transfer of ultrasound technology.

At the same time, mobile “personal” terminal devices (e.g. smartphones, tablets, etc.) have experienced a strong market penetration in recent years. Driven by the broad mass market in the so-called “consumer sector”, both the performance of these devices and their possibilities of use increase. This development has been recognized by various manufacturers for several years and first devices based on consumer electronics are realized. However, these devices are always designed for a specific use case, are relatively large and only use the terminal device as a compact user interface or as an interface to a cloud.

In summary, it should be noted that existing devices each cover only a specific or very narrow field of use and only a few are usable in a mobile manner.

Against this background, one task is to enable an improved use and more flexible provision of ultrasound.

### ABSTRACT

According to the invention, the task is solved by a modular ultrasonic device, an ultrasonic system, and a method for modifying a modular ultrasonic device according to the independent claims. Further aspects and further developments of the invention are described in the dependent claims, the following description and in the figures.

A first embodiment relates to a modular ultrasonic device. The modular ultrasonic device comprises a housing and at least one ultrasonic transducer. The at least one ultrasonic transducer is configured to generate and emit ultrasonic waves based on a respective control signal and to generate a respective measurement signal depending on received ultrasonic waves. The modular ultrasonic device further comprises a plurality of boards arranged in the housing, which are separably connected to one another via respective plug connectors. The plurality of boards comprise at least one first board with a power supply circuit configured to generate a respective power supply signal for the further boards of the plurality of boards. The plurality of boards further comprise at least one transmit circuit configured to generate the respective control signals for the at least one ultrasonic transducer, and a receive circuit configured to process the respective measurement signals of the at least one ultrasonic transducer. The transmit circuit and the receive circuit are either both formed on a second board of the plurality of boards or the transmit circuit is formed on the second board and the receive circuit is formed on a third board of the plurality of boards. The at least one ultrasonic transducer is formed on a fourth board of the plurality of boards or is separably connected to one of the plurality of boards.

A second embodiment relates to an ultrasonic system. The ultrasonic system comprises a plurality of modular ultrasonic devices according to the invention and a device for evaluating measurement data. The plurality of modular ultrasonic devices are configured to transmit respective measurement data directly or by forwarding via at least one further of the plurality of modular ultrasonic devices to one or more predetermined modular ultrasonic devices of the plurality of modular ultrasonic devices. The one or more predetermined modular ultrasonic devices are configured to transmit the collected measurement data of the plurality of modular ultrasonic devices to the device for evaluating measurement data. The device for evaluating measurement data is configured to determine one or more predetermined characteristics based on the measurement data.

A third embodiment relates to a method for modifying a modular ultrasonic device according to the invention. The method comprises non-destructively separating one of the plurality of boards from the remaining boards of the plurality of boards by detaching at least a part of the plug connectors. In addition, the method comprises separably connecting a new board to the remaining boards of the plurality of boards by means of one or more plug connectors.

### BRIEF DESCRIPTION OF THE FIGURES

Some examples of devices and/or methods will be described in the following by way of example only and with reference to the accompanying figures, in which:

FIG. 1 shows a three-dimensional illustration of a first embodiment of a modular ultrasonic device;

FIG. 2 shows an exploded view of the modular ultrasonic device shown in FIG. 1;

FIG. 3 shows a schematic illustration of a second embodiment of a modular ultrasonic device;

FIG. 4 shows a schematic illustration of a third embodiment of a modular ultrasonic device;

FIG. 5a and FIG. 5b show a schematic illustration of a fourth embodiment of a modular ultrasonic device which is coupled to additional devices;

FIG. 6 shows a schematic illustration of a first embodiment of an ultrasonic system;

FIG. 7 shows a schematic illustration of a second embodiment of an ultrasonic system;

FIG. 8 shows a schematic illustration of a third embodiment of an ultrasonic system;

FIG. 9 shows an application scenario for the ultrasonic system shown in FIG. 8; and

FIG. 10 shows a flow diagram of an embodiment of a method for modifying a modular ultrasonic device.

### DESCRIPTION

Some examples are now described in more detail with reference to the enclosed figures. However, other possible examples are not limited to the features of these embodiments described in detail. These may include modifications of the features as well as equivalents and alternatives to the features. Furthermore, the terminology used herein to describe certain examples should not be restrictive of further possible examples.

Throughout the description of the figures, same or similar reference numerals refer to same or similar elements and/or features, which may, in each case, be identical or implemented in a modified form while providing the same or a similar function. The thickness of lines, layers and/or areas in the figures may also be exaggerated for clarification.

When two elements A and B are combined using an ‘or’, this is to be understood as disclosing all possible combinations, i.e., only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a,” “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not exclude the presence or addition of one or more other features, integers, steps, operations, processes, elements, components and/or a group thereof.

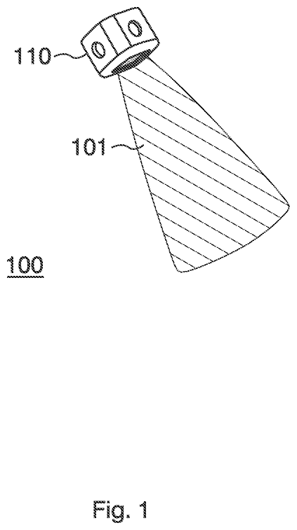

FIG. 1 shows a modular ultrasonic device 100 according to the present disclosure. The modular ultrasonic device 100 comprises a housing 110, in which the various components of the modular ultrasonic device 100 are accommodated. The modular ultrasonic device 100 is configured to emit ultrasonic waves 101.

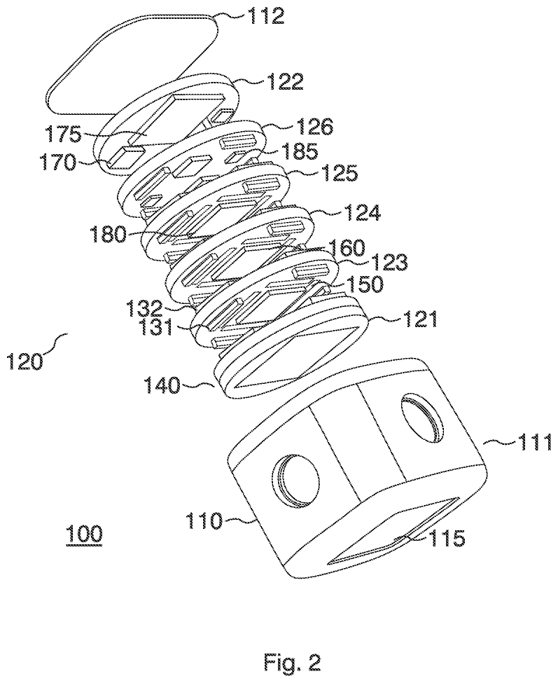

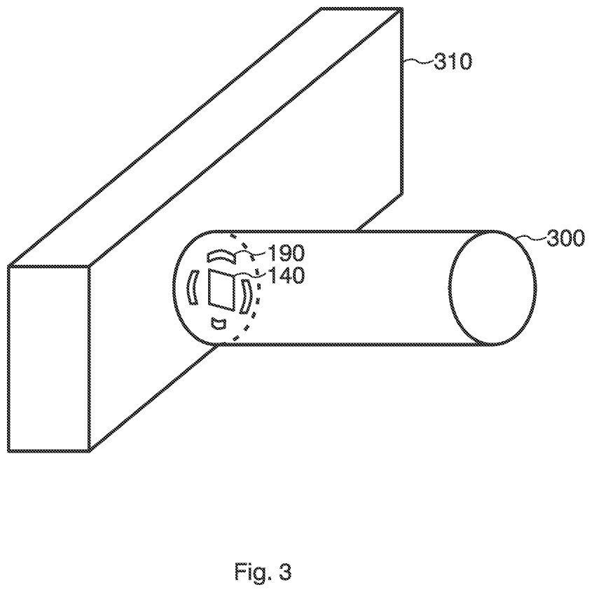

An exploded view of the modular ultrasonic device 100, in which the individual components of the modular ultrasonic device 100 can be seen, is shown in FIG. 2. The housing 110 of the modular ultrasonic device 100 consists of two parts 111 and 112, which are connectable to one another and non-destructively separable from one another, so that the various components are accessible and/or reachable within the modular ultrasonic device 100. However, it should be noted that the housing 110 of the modular ultrasonic device 100 does not necessarily have to consist of two parts. According to the present disclosure, the housing 110 may also consist of more than two parts, which are connectable to one another and nondestructively separable from one another. In the embodiment of FIGS. 1 and 2, the housing 110 has a substantially cubic shape with rounded edges. However, it should be noted that the housing 110 of the modular ultrasonic device 100 does not necessarily have to have a substantially cubic shape with rounded edges. In principle, the housing 110 may have any suitable shape (see e.g. the embodiment of FIG. 3).

As can be seen from FIG. 2, the modular ultrasonic device 100 comprises a plurality of boards 120, which are arranged in the housing 110. The plurality of boards 120 are separably connected to one another via respective plug connectors 131, 132. The part 112 of the housing 110 may be designed e.g. as a housing rear side with a mechanism for closing (e.g. screw or clamping mechanism) in order to be able to reach the plurality of boards 120 and/or to introduce them into the housing 110.

The modular ultrasonic device 100 comprises at least one ultrasonic transducer 140, which is configured to generate and emit the ultrasonic waves 101 based on a respective control signal. Furthermore, the at least one ultrasonic transducer 140 is configured to generate a respective measurement signal depending on received ultrasonic waves. In the embodiment of FIG. 2, the at least one ultrasonic transducer 140 is formed on a first board 121 of the plurality of boards 120. According to some embodiments, the first board 121 may also have a plurality of ultrasonic transducers 140 (i.e. two or more) configured as described above.

In general, different geometries and arrangements of acoustic elements serving as ultrasonic transducers (e.g. single-element transducers with and without structured lenses for volume imaging, array transducers in linear, convex, concave, T- and matrix geometry or any shape and element arrangement) with different properties (narrow-band, broad-band, sensitive) as transmitter or as receiver or both or only partially may be implemented and combined. Thus, parameters such as the application-specific frequency range, the output power, the number of channels, the ultrasonic geometry, etc. may be varied very simply and flexibly via the standardized hardware interfaces. A wide frequency range may be realized (Hz, kHz, to MHz range) from audible sound to airborne sound to structure-borne sound from liquids to solids. With this range of types of possible ultrasonic transducers, a wide range of point-by-point measurements, simple imaging up to volumetric acquisition may thus be carried out.

The first board 121 may be considered as an ultrasonic transducer module of the modular ultrasonic device 100. However, it should be noted that the at least one ultrasonic transducer 140 does not have to be and/or not every ultrasonic transducer in the case of a plurality of ultrasonic transducers needs to be formed on one of the plurality of boards 120. In alternative embodiments, the at least one ultrasonic transducer 140 may also be arranged separately from the plurality of boards 120 in the housing 110 and, for example, separably connected to one of the plurality of boards 120 (e.g. via one or more cables).

The housing 110 comprises a window with increased acoustic transparency compared to the rest of the housing. The window 115 is arranged in front of the at least one ultrasonic transducer 140. In the example of FIG. 2, the window 115 is formed by a recess (opening) in the housing 110. However, it should be noted that the window 115 does not necessarily have to be formed by a recess in the housing 110. Rather, the housing may also be formed in a partial region in front of the at least one ultrasonic transducer 140 from a first material or a first material mixture, which has an adapted or increased acoustic transparency compared to a second material or a second material mixture of the rest of the housing.

Furthermore, the plurality of boards 120 comprises at least one second board 122 with a power supply circuit 170 configured to generate a respective power supply signal for the at least one ultrasonic transducer and the further boards of the plurality of boards 120. Furthermore, the power supply circuit 170 may be configured for the energy management of the modular ultrasonic device 100 as required. For example, the power supply circuit 170 may be configured to place one or more of the plurality of boards 120 or individual components on the plurality of boards at least partially into an energy-saving mode (for example, a sleep mode) during, for example, long-term measurement tasks. The power supply circuit 170 or another circuit (for example, a processing circuit described in more detail below) may additionally adaptively parameterize the sleep mode autonomously depending on previous measurements and the results thereof, in order to carry out long-term measurements even more efficiently. One factor on which this may be made dependent, for example, is the early reaching of a limit value to be monitored for shortening measurement intervals (for example, maximum or minimum filling level) or the extension of the measurement interval if the measurement value is far from critical limits.

Optionally, the second board 120 may further comprise an accumulator 175 coupled to the power supply circuit 170. Accordingly, the power supply circuit 170 may be configured to generate the respective power supply signal based on energy stored in the accumulator 175. The accumulator 175 may also be designed to be exchangeable. For example, the storage capacity of the accumulator 175 may be selected depending on the intended use and application. The power supply circuit 170 also assumes charge management for the accumulator 175 (depending on the selected energy source, see below).

The modular ultrasonic device 100 may optionally comprise a plurality of second boards 122 in order to increase the total capacity of the available electrical energy. The total capacity of the available electrical energy is then not defined by the capacity of the accumulator on the individual board, but is scalable by using a plurality of second boards 122. For example, only the power supply circuit 170 of an individual one of the plurality of second boards may be integrated with the above-described charging function and may be configured to charge the accumulators of the further second boards, for example, by means of inductive coupling.

Alternatively or additionally, a socket (not illustrated in FIGS. 1 and 2) for connection to a charging cable may be integrated into the housing 110. Accordingly, the power supply circuit 170 may be configured to generate the respective power supply signal based on electrical energy received at the socket. The socket may be designed both proprietary and in accordance with an industry standard (for example, high-level sockets for using widely used plug and table power supplies). The power supply may also be “bus-powered”, wherein the system is supplied with electric power in parallel from the outside via an inserted communication channel. This may be done, for example, by means of Power over Ethernet (PoE) when using Local Area Network (LAN)/Ethernet or via Universal Serial Bus (USB), wherein the respective data cable simultaneously functions as a charging cable.

Further alternatively or additionally, the modular ultrasonic device 100 may comprise an energy converter (not illustrated in FIGS. 1 and 2) configured to convert ambient energy from the surroundings of the modular ultrasonic device 120 into electrical energy. Accordingly, the power supply circuit 170 may be configured to generate the respective power supply signal based on the electrical energy provided by the energy converter. For example, the energy converter may be provided in the form of a photoelectric converter attached to the housing, such as one or more solar cells. Alternatively or additionally, the energy converter may be provided in the form of a thermoelectric converter (e.g. using the Seebeck effect, the Peltier effect and/or the Thompson effect). Further alternatively or additionally, the energy converter may be provided in the form of one or more coils in order to convert electromagnetic energy provided in the surroundings of the modular ultrasonic device 120 into electrical energy by means of induction (e.g. in accordance with a proprietary energy transmission or in accordance with an industry standard such as “Qi”). Alternatively or additionally, the energy converter may be provided in the form of an electromechanical converter (e.g. using the Piezo effect) in order to convert mechanical energy present in the surroundings of the modular ultrasonic device 120 (e.g. oscillations, vibrations, pressures or ultrasonic waves) into electrical energy. In this way, a complete or additionally wireless power supply of the modular ultrasonic device 100 may be enabled. As shown in FIG. 2, the second board 122 may be e.g. the last board of the plurality of boards 120 and the housing part 112 may be adapted to the energy converter in order to enable an optimal utilization of the ambient energy.

As the above examples have shown, the supply with electrical energy may be carried out in a wired and/or wireless manner. Both direct current and alternating current sources may be used. In the case of an alternating current source, for example, the power supply circuit 170 may carry out the necessary rectification of the alternating current into direct current in order to be able to supply the electronics of the modular ultrasonic device 100 with direct current.

The modular ultrasonic device 100 further comprises at least one transmit circuit 150, which is configured to generate the respective control signals for the at least one ultrasonic transducer 140, and a receive circuit 160, which is configured to process the respective measurement signals of the at least one ultrasonic transducer 140. In the embodiment of FIGS. 1 and 2, the transmit circuit 150 is formed on a third board of the plurality of boards 120 and the receive circuit is formed on a fourth board 124 of the plurality of boards 120. In other words: The transmit circuit 150 and the receive circuit 160 are arranged on different boards. However, it should be noted that the transmit circuit 150 and the receive circuit 160 do not necessarily have to be arranged on different boards. In alternative embodiments, the transmit circuit 150 and the receive circuit 160 may also be arranged on the same board. For example, the transmit circuit 150 and the receive circuit 160 may be arranged together on the third board 123. If the transmit circuit 150 and the receive circuit 160 are arranged on the same board, the transmit circuit 150 and the receive circuit 160 may also be configured as an integrated circuit, i.e. as a transceiver circuit.

The transmit circuit 150 is configured to generate the electrical transmission patterns and pulses for driving the at least one ultrasonic transducer 140 and to bring them to a corresponding voltage level. This may comprise at least one of phase, frequency, amplitude and pulse width modulations. The respective control signal for the at least one ultrasonic transducer 140 accordingly indicates the electrical transmission patterns and pulses for driving the at least one ultrasonic transducer 140. The third board 123 with the transmit circuit 150 may be considered as an ultrasonic electronics module for transmission.

The receive circuit 160 is configured to process and/or pre-process the respective measurement signal of the at least one ultrasonic transducer 140. For this purpose, the receive circuit 160 may at least be configured to at least amplify and digitize the respective measurement signal of the at least one ultrasonic transducer 140. Optionally, the receive circuit 160 may also be configured to further process the respective measurement signal of the at least one ultrasonic transducer 140. For example, the receive circuit 160 may further be configured to compress, filter, add the respective measurement signal of the at least one ultrasonic transducer 140 to another signal (e.g. add the measurement signals of multiple ultrasonic transducers). The fourth board 124 with the receive circuit 160 may be considered as an ultrasonic electronics module for reception.

A configuration of the plurality of boards 120, which includes at least the first board 121, the second board 122, as well as the transmit circuit 150 and the receive circuit 160—regardless of whether they are arranged on a common board or separate boards—may be considered as a basic configuration of the proposed modular ultrasonic device. All further components and/or functionalities of the modular ultrasonic device described above and below are optional. Accordingly, if the at least one ultrasonic transducer 140 is not configured on one of the plurality of boards 120, the at least one ultrasonic transducer 140, together with a configuration of the plurality of boards 120, which includes at least the second board 122, as well as the transmit circuit 150 and the receive circuit 160—regardless of whether they are arranged on a common board or separate boards—may be considered as a basic configuration of the proposed modular ultrasonic device. The at least one ultrasonic transducer 140 may, for example, be separably connected to the board(s) on which the transmit circuit 150 and the receive circuit 160 are configured (e.g. by means of one or more cables).

For example, the plurality of boards may further comprise a fifth board 125 with a processing circuit 180. The processing circuit 180 is configured to recognize a respective functionality of the plurality of boards 120 and, based thereon, to control a data exchange between at least a part of the plurality of boards 120 (or all of the plurality of boards 120, if applicable). The modular ultrasonic device 100 is controlled by the processing circuit 180. Recognition and control of the individual modules and/or boards, as well as control of the communication and data exchange of the modules and/or boards among one another, may take place in a variety of ways and is known per se in each case. Therefore, this will not be discussed in more detail at this point.

For example, the entire configuration of the modular ultrasonic device 100 may be configured into the modules and/or boards during the startup and production of the device. In this way, the existing components are known to the individual modules and/or boards, and faults or failures of other modules may be detected by the remaining modules. In this way, by means of one or more emergency operating modes, if applicable, a further measurement operation may be continued by the modular ultrasonic device 100 itself, despite partial defect and failure, by automatic reconfiguration.

For the processing circuit 180, differently powerful and thus differently cost-intensive hardware may be used—depending on the desired application of the modular ultrasound device 100. Thus, the fifth board 125 may be adapted to the required performance. For example, the processing circuit 180 may be formed by and/or comprise a processor, a computer processor (CPU=Central Processing Unit), a microcontroller, an application-specific integrated circuit (ASIC), an integrated circuit (IC), a system on a chip (SoC), a programmable logic element or a field programmable gate array (FPGA) with a microprocessor on which software for controlling the one and/or more constituents of the modular ultrasonic device 100 runs according to the principles described herein. Furthermore, the control circuit may have and/or be coupled to one or more memories.

For example, the processing circuit 180 may be designed to be more powerful in order, for example, to carry out data processing steps, to carry out data analyses or to use artificial intelligence (AI) networks. In other words: The processing circuit 180 may optionally be configured to determine one or more predetermined characteristics based on the respective measurement signal of the at least one ultrasonic transducer 140 processed by the receive circuit 160. In particular, the processing circuit 180 may be configured to determine the one or more predetermined characteristics using a model trained by machine learning. However, no model trained by machine learning has to be used for this purpose. The machine learning may be carried out, for example, on radio-frequency (RF) data such as are present directly after the ultrasonic digitization by the receive circuit 160. For processing the ultrasonic data, these may be regarded as time series data, for example. For the processing, the classification may take place in the sense of a “time series classification”. For this purpose and for other types of further processing, the determination and/or extraction of characteristics (features) may use temporal, spectral, and statistical methods, for example.

The fifth board 125 may be considered as an ultrasonic electronics module with control logic.

The processing circuit 180 may also function as a multiplexer module in order to control a programmable, dynamic, and/or fixed connection between individual elements of the first board 121 (e.g., individual ultrasonic transducers when a plurality of ultrasonic transducers is arranged on the first board 121) and electronic channels of the transmit circuit 150 and/or of the receive circuit 160.

The plurality of boards 120 may comprise a sixth board 126 having a data memory 185, for example. The data memory 185 is configured to store the respective measurement signal of the at least one ultrasonic transducer 140 after processing by the receive circuit 160. The data memory 185 may be used for storing measurement data. The data memory 185 may either be fixedly integrated on the sixth board 126 or may also be designed to be exchangeable. The data memory 185 may comprise both volatile memory (e.g., random access memory, RAM) and persistent memory such as a secure digital (SD) card, a micro-SD card, a flash memory, a solid-state drive (SSD), a multimedia card (MMC), a memory connected via a USB interface (e.g., a USB stick), or a hard disk drive (HDD). The sixth board may be considered as a data memory module. Optionally, the data memory 185 may also be arranged together with the processing circuit 180 on a common board.

Likewise, the plurality of boards 120 may further comprise a seventh board (not illustrated in FIGS. 1 and 2) having a position sensing circuit. The position sensing circuit is configured to determine an absolute position of the modular ultrasonic device 100 and/or a relative position of the modular ultrasonic device with respect to a predetermined object. Depending on the requirement, this board may determine the position and, if applicable, location of the modular ultrasonic device 100 absolutely on a small or global scale or relative, for example, to one another in a sensor network. The absolute, own position sensing may be carried out, for example, via a receiver for a global navigation satellite system such as, for example, NAVSTAR GPS or Galileo, the localization may be carried out on the basis of wireless local area networks (WLAN) and also via local position sensing systems (e.g. acceleration and/or position sensors, i.e. inertial sensors, optical, acoustic or also other sensors). The relative positioning of the modular ultrasonic device 100 in a sensor network or in the case of connected individual devices may take place, for example, by means of fixed mechanical interfaces, by means of mechanical sensor technology such as sensor strips (stretching, bending, etc.), by means of external position sensor technology (for example optically by image processing, optically by tracking such as infrared tracking), by means of radio reference points (for example Bluetooth beacons) or by the ultrasonic sensor technology itself (for example triangulation by means of ultrasonic signals in airborne sound or immersion sound depending on the surrounding medium). The seventh board may be considered as a position sensing module.

Optionally, the modular ultrasonic device 100 may further comprise a user interface (not illustrated) integrated into the housing. The user interface may be configured, for example, to optically and/or acoustically output information relating to the modular ultrasonic device 100 to a user. Alternatively or additionally, the user interface may be configured to receive a user input of the user. The user interface may be considered as a display and interaction module. The user interface may be used, for example, for displaying status or measurement values and user interaction. The user interface may comprise, for example, a display for simple numerical value output, or else a screen for graphically outputting an ultrasound image or a curve profile. One variant may also be designed as a touchscreen for receiving user inputs. Likewise, the user interface may comprise other mechanical input options such as buttons, pushbuttons, keys, etc., in order to enable user inputs. Alternatively or additionally, one or more loudspeakers for the information output and also one or more microphones for receiving user inputs from the user interface may be comprised.

According to embodiments, the plurality of plug connectors 131, 132 may be configured to conduct the respective power supply signal from the second board 122 to the respective further board of the plurality of boards 120. In other words: the general power supply of the individual boards may take place via the plurality of plug connectors 131, 132. Alternatively, the respective power supply signal from the second board 122 to one or more (e.g. to all) of the plurality of boards 120 may take place via another interface. For example, both the second board 122 and one or more (e.g. all) of the plurality of boards 120 may each comprise one or more coils in order to conduct the respective power supply signal inductively from the second board 122 to the respective further board of the plurality of boards 120.

Similarly, the plurality of plug connectors may comprise signal paths for the exchange of data between at least a part of the plurality of boards. In other words: the communication between the individual boards may take place via the plurality of plug connectors 131, 132. Alternatively, the communication between individual boards of the plurality of boards 120 may also take place via another interface. For example, individual boards of the plurality of boards 120 may comprise transmitters and/or receivers for a corresponding wireless communication. For example, Bluetooth, near-field communication NFC), WLAN, optical communication or communication by means of sound waves may be used. Independently of the specific type of data exchange, the individual components on the plurality of boards may exchange data with one another via a communication bus.

The individual modules and/or boards are-as explained above-connected to one another via the plug connectors 131, 132 or other interfaces (e.g. inductively, Bluetooth or others) via a corresponding bus system. In this case, all of the modules and/or boards described above and below do not necessarily have to be present, in particular, they do not have to be arranged in a specific sequence. In preferred embodiments, the plug connectors 131, 132 contain both the general power supply and data and communication bus systems for the functional communication. Both serial and parallel communication may take place on the buses.

For the communication with external devices, the plurality of boards 120 may further comprise an eighth board (not illustrated in FIGS. 1 and 2) having a transceiver circuit. The transceiver circuit is configured to generate a transmit signal based on data of the mobile ultrasonic device to be transmitted and accordingly to determine receive data for the mobile ultrasonic device based on a receive signal.

The transmit signal may be output in a variety of ways. For example, the transceiver circuit may be configured to apply the transmit signal to an antenna (not illustrated in FIGS. 1 and 2) of the modular ultrasonic device 100 for emission into surroundings of the modular ultrasonic device 100. The antenna may, for example, be integrated into the housing 110 or be arranged on the eighth board or a separate board. Alternatively or additionally, the transceiver circuit may be configured to apply the transmit signal to an interface (not illustrated in FIGS. 1 and 2) of the mobile ultrasonic device for wired communication with an external device. Further alternatively or additionally, the transceiver circuit may be configured to forward the transmit signal to the transmit circuit 150 in order to generate the respective control signal for the at least one ultrasonic transducer 140 based on the transmit signal and thus to encode the data of the mobile ultrasonic device 100 to be transmitted into the ultrasonic waves 101 emitted by the at least one ultrasonic transducer 140.

In an analogous manner, the receive signal may be received in a variety of ways. For example, the transceiver circuit may be configured to receive the receive signal from the antenna. Alternatively or additionally, the transceiver circuit may be configured to receive the receive signal from the interface for wired communication with the external device. Further alternatively or additionally, the transceiver circuit may be configured to receive the receive signal from the receive circuit 160, wherein the receive circuit 160 is configured to derive the receive signal from the respective measurement signal of the at least one ultrasonic transducer 140.

The eighth board may be considered as a communication module.

For the communication and for the data transfer between the modular ultrasonic device 100 and external hardware such as, for example, a mobile terminal device or a further modular ultrasonic device, a standardized communication protocol may be used, for example. In this way, the modular ultrasonic device 100 may be connected in a simple way to any desired terminal device, backend, etc. depending on the requirement.

As the above explanations have shown, both wired (e.g. wired or fiber-optical) and cordless (wireless) interfaces may be used. Here, in principle, both proprietary interfaces and/or communication protocols and standardized interfaces and/or communication protocols may be used. For the wired communication, for example, simple serial interfaces such as RS-232 may be used natively or serially via USB, the native USB interface (e.g. USB 2.0/3.X/4), special industrial bus systems, manufacturer-specific interface (e.g. USB via Lightning), LAN, CAN bus or Mil bus. For the wireless communication, for example, WLAN, Bluetooth, simple RF radio interfaces (e.g. 433 MHz/866 MHz radio), ZigBee (Z-Wave), LoRaWAN and mobile radio standards such as 2G, 4G and 5G or future standards may be used.

It has already been indicated above that the modular ultrasonic device 100 may have multiple communication interfaces. In this way, the modular ultrasonic device 100 (e.g., the processing circuit 180 or the transceiver circuit of the eighth board) may dynamically decide which interface and/or which interfaces are used. If a remote station is present in the vicinity, an interface with a high bandwidth may be used, for example. Without this, a long-range radio interface with possibly lower bandwidth may be used. This enables, for example, the automatic switch between autonomous operation with remote transmission and local service or local readout by a technician on site. With a plurality of integrated communication interfaces, the modular ultrasonic device 100 may also automatically recognize whether a communication channel is defective or disrupted and consequently use another available channel/system.

As already indicated above, in addition to a dedicated communication system according to some embodiments, the modular ultrasonic device 100 may also communicate itself by means of sound or ultrasound. When using ultrasound by, for example, a plurality of modular ultrasonic devices in a common medium (for example, the same space when using airborne sound or the same liquid when immersed), in addition to the measurement task, the generation and detection of ultrasound may also be used in order, in addition to the actual measurement task, also to exchange information (for example, measurement parameters or results) between the modular ultrasonic devices themselves.

According to embodiments, the formatting of the digital communication may use interface formats for data transmission such as JSON, XML, or REST service in addition to classical binary-formatted serial transmissions.

In the embodiment of FIGS. 1 and 2, the plurality of boards 120 are arranged one above the other and successive boards of the plurality of boards 120 are respectively plugged together correspondingly by means of the plug connectors 131, 132. In this way, the plurality of boards 120 may be arranged compactly in the form of a stack. However, it should be noted that the plurality of boards 120 do not necessarily have to be arranged one above the other.

In alternative embodiments, the plurality of boards may be arranged next to one another and/or laterally offset with respect to one another. For example, the further boards of the plurality of boards 120 may be plugged onto the first board 121 by means of the plug connectors 131, 132. Alternatively, the plurality of boards 120 may also be plugged onto a main board by means of the plug connectors 131, 132. Likewise, the plurality of boards 120 may be arranged such that they only partially overlap one another. The shape of the housing 110 may be adapted accordingly. For this purpose, rotationally symmetrical plug connectors may be used, for example, or a recognition of the rotation is carried out by detection of a pin assignment at the plug connector.

The housing 110 may be a modular housing which, depending on the application, may be coated with cost-effective casings (e.g. made of injection-molded plastic or 3D printing). For the same internal dimensions of the electronics to be enclosed, in the case of harsher ambient conditions or (mobile) use conditions, however, rugged, dustproof and/or waterproof housings may also be selected. Likewise, the housing 110 may be designed for particularly high temperatures or pressures in order to be usable, for example, for applications in the industrial environment. Housing variants made of metal may additionally be used in order to facilitate a heat dissipation of the electronic components in the interior of the housing 110 by, for example, individually adapted heat-conducting constructions (e.g., “heat pipes”) of particularly heat-generating modules and/or boards themselves being integrated into the housing 110.

Optionally, a fastening element (not illustrated in FIGS. 1 and 2) for fastening the modular ultrasonic device 100 to a test object, i.e. an object to be tested by means of the modular ultrasonic device 100, may be integrated into the housing 110. The fastening element may be configured in a variety of ways. The fastening element may be, for example, a frame for accommodating adhesive pads in order to adhere the modular ultrasonic device 100 to the test object. Alternatively, the fastening element may be, for example, a frame with eyelets for one or more straps, one or more hose clamps or a holder with eyelets for cable ties or the like.

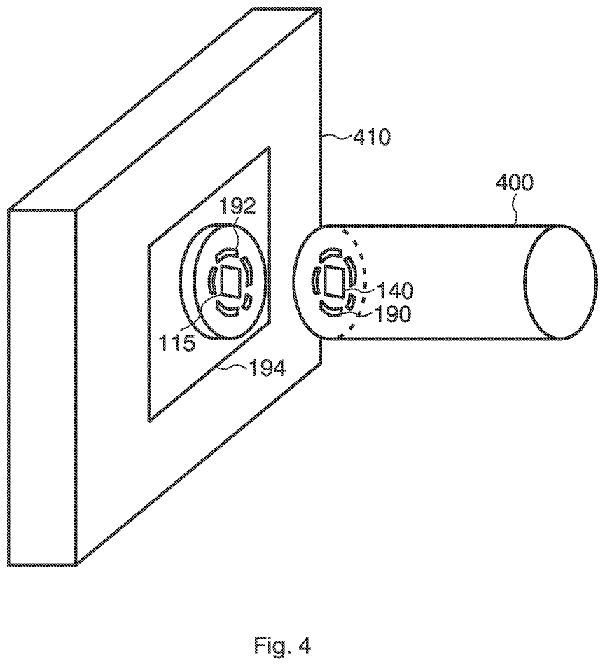

Two possible embodiments of the fastening element will be explained in more detail below with reference to FIGS. 3 and 4.

FIG. 3 shows a further modular ultrasonic device 300 configured according to the above explanations. As can be seen from a comparison of FIG. 1 and FIG. 3, the housing of the modular ultrasonic device 300, in contrast to the housing of the modular ultrasonic device 100, does not have a cube shape, but is configured in an elongated manner-similar to a rod.

Furthermore, a magnetic test object (test item) 310 such as a metallic (wall) surface, a tube or another metallic surface is shown in FIG. 3. The modular ultrasonic device 300 is positioned on the test object 310 via a magnetic holder in the front area of the modular ultrasonic device 300. For this purpose, the fastening element of the modular ultrasonic device 300 comprises one or more magnets 190, which are arranged such that they do not obstruct the at least one ultrasonic transducer 140 of the modular ultrasonic device 300. For example, the one or more magnets 190 may be integrated into the housing of the modular ultrasonic device 300 or the first board, which carries the at least one ultrasonic transducer 140. The proposed magnetic holder enables a direct connection/positioning of the modular ultrasonic device 300 to a metallic test item, for example. If applicable, a dry coupling pad or a coupling medium between the magnetic holder and the test object may additionally be used.

Although a fastening of the modular ultrasonic device 300 on a substantially planar surface of the test object 310 is shown in FIG. 3, it should be noted that both the front area of the modular ultrasonic device 300 and the counterpart in the shape of the test object may have any desired shape. For example, the test object 310 may also be formed in a ring shape and the front area of the modular ultrasonic device 300 may be adapted accordingly.

It should also be noted that the magnetic holder for certain applications may also be attached in an analogous manner in the rear area and/or a rear side of the modular ultrasonic device 300. This may enable a simple mounting, for example for airborne sound applications, on metallic surfaces.

FIG. 4 further shows an alternative embodiment of the fastening element for a non-magnetic test object 410. In the example of FIG. 4, a magnetic adhesive adapter is used as the fastening element in order to fasten the modular ultrasonic device 300 to the non-magnetic test object 410.

For this purpose, the fastening element of the modular ultrasonic device 400 consists of two parts that are connectable to one another and/or separable from one another. The first part of the fastening element is formed by one or more magnets 190, which are arranged such that they do not obstruct the at least one ultrasonic transducer 140 of the modular ultrasonic device 300. For example, the one or more magnets 190 may be integrated into the housing of the modular ultrasonic device 400 or the first board, which carries the at least one ultrasonic transducer 140. The second part of the fastening element is formed by an adhesive surface or otherwise mechanically attachable surface 194, on which one or more counter magnets 194 are formed and/or arranged, which may magnetically couple to the one or more magnets 190 in order to fasten the modular ultrasonic device 400 to the adhesive surface 194 and to the test object 410 by means of adhering the adhesive surface 194 to the test object 410. As illustrated in FIG. 4, the one or more counter magnets 194 may be held in a housing portion, which may terminate flush with the remaining housing of the modular ultrasonic device 400 in order to enable an optimal positioning of the modular ultrasonic device 400 on the test object 410.

In the example of FIG. 4, a counterpart is thus used for the reproducible positioning of the modular ultrasonic device 400, which itself is magnetic and is applied to the test object 410 by means of an adapter (e.g. adhesive surface). The magnetic adhesive adapter may be exactly fitting by integrating individual magnets in an unambiguous positioning, which offer an encoding of the magnet positions.

The magnetic fastening both on the transducer front of the modular ultrasonic device on the part of a test object and on the rear side for mounting the modular ultrasonic device may, if applicable, be reinforced by electromagnetic function during operation. Here, too, a counterpart may, if applicable, be necessary and used if the test object to which the modular ultrasonic device is to be fastened, does not have a metallic and magnetic surface.

The hardware of the modular ultrasonic devices described above and below represents a variable plug-in modular system, which consists of individual modules and/or boards matched to one another.

The respective housing of the modular ultrasonic devices described above and below extends, independently of its specific shape, three-dimensionally in space, i.e. in three spatial directions perpendicular to one another. The modular ultrasonic devices proposed herein may be configured very compactly, so that they may, for example, be accommodated comfortably in a human hand and be moved by the same over a test object. For example, an extension of the housing in each of the three spatial directions perpendicular to one another may be less than 15 cm, 10 cm or 5 cm.

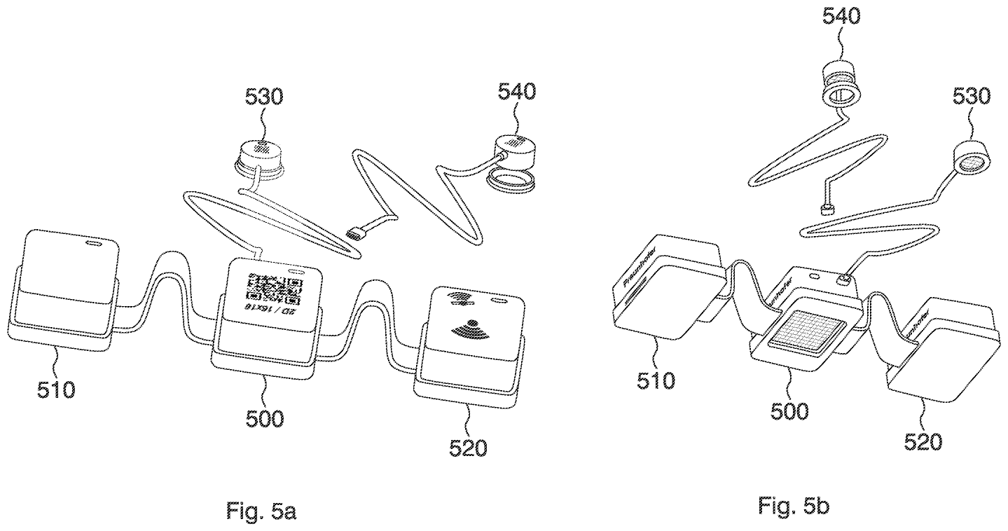

FIG. 5a and FIG. 5b show a perspective view of a further modular ultrasonic device 500 which is coupled to additional devices 510, 520, 530 and 540.

The modular ultrasonic device 500 is configured according to the basic configuration described above. In other words: The modular ultrasonic device 500 comprises, in addition to the at least one ultrasonic transducer, the transmit circuit, the receive circuit and the power supply circuit. In the additional device 510, to which the modular ultrasonic device 500 is coupled by cable, an accumulator is provided which provides the electrical energy for the power supply circuit. In the additional device 520, to which the modular ultrasonic device 500 is coupled by cable, the transceiver circuit described above is provided, so that the modular ultrasonic device 500 may communicate with external devices via the additional device 520. In addition, the modular ultrasonic device 500 is connected by cable to two further additional devices 530 and 540 which have further ultrasonic transducers. The transmit circuit and the receive circuit may output respective control signals to the further ultrasonic transducers of the additional devices 530 and 540 via the cables and accordingly receive measurement signals of the further ultrasonic transducers of the additional devices 530 and 540.

The functionality of the modular ultrasonic device 500 may be extended by means of the additional devices 510, 520, 530 and 540.

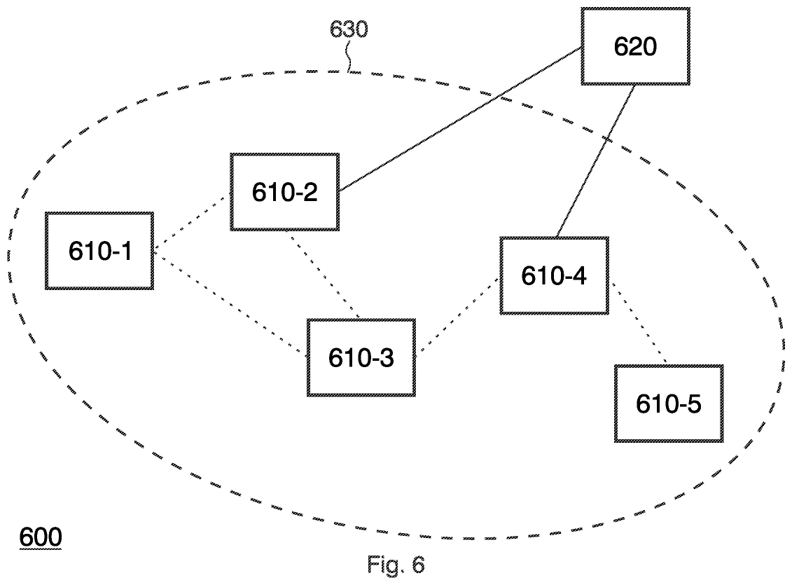

After the details of different modular ultrasonic devices according to the present disclosure have been described above, some ultrasonic systems which use modular ultrasonic devices according to the present disclosure will be described below with reference to FIGS. 6 to 9.

FIG. 6 shows an ultrasonic system 600 which, in addition to a device 620 for evaluating measurement data, also has five modular ultrasonic devices 610-1, . . . , 610-5 according to the present disclosure. Although exactly five modular ultrasonic devices are shown in the embodiment of FIG. 6, it is self-evident that the present disclosure is not limited thereto and an ultrasonic system according to the present disclosure may in principle comprise any desired plurality of modular ultrasonic devices according to the present disclosure.

The modular ultrasonic devices 610-1, . . . , 610-5 are each configured at least according to the basic configuration described above and each additionally comprise the eighth board described above with the transceiver circuit. The modular ultrasonic devices 610-1, . . . , 610-5 form a sensor network and/or mesh 630.

The modular ultrasonic devices 610-1, . . . , 610-5 are configured to transmit respective measurement data directly or by forwarding via at least one further of the modular ultrasonic devices 610-1, . . . , 610-5 to one or more predetermined modular ultrasonic devices of the ultrasonic devices 610-1, . . . , 610-5. In the example of FIG. 6, the modular ultrasonic devices 610-2 and 610-4 are predetermined ultrasonic devices. As can be seen from FIG. 6, the modular ultrasonic device 610-1 transmits its measurement data directly to the predetermined ultrasonic device 610-2 and via the ultrasonic device 610-3, which serves as a relay station, to the predetermined ultrasonic device 610-4. The modular ultrasonic device 610-3 transmits its measurement data directly to the predetermined modular ultrasonic devices 610-2 and 610-4. The modular ultrasonic device 610-3 transmits its measurement data directly to the predetermined ultrasonic device 610-4.

The predetermined modular ultrasonic devices 610-2 and 610-4 are configured to transmit the collected measurement data of the modular ultrasonic devices 610-1, . . . , 610-5 to the device 620 for evaluating measurement data. The device 620 for evaluating measurement data is in turn configured to determine one or more predetermined characteristics based on the received measurement data. The one or more predetermined characteristics may be determined as described above. For example, the device for evaluating measurement data may be configured to determine the one or more predetermined characteristics from the measurement data using a model trained by machine learning. For the determination of the one or more predetermined characteristics, the device 620 for evaluating measurement data may have a corresponding processing circuit such as, for instance, a processor, a CPU, an ASIC, an IC, an SoC, an FPGA with a microprocessor or a more complex local computing cluster on which software for the determination of the one or more predetermined characteristics runs according to the principles described herein. Furthermore, the processing circuit of the device 620 for evaluating measurement data may have one or more memories and/or be coupled thereto.

As indicated by the dashed lines between the modular ultrasonic devices 610-1, . . . , 610-5 in FIG. 6, the modular ultrasonic devices 610-1, . . . , 610-5 may be configured, for example, to transmit the measurement data to the predetermined modular ultrasonic devices 610-2 and 610-4 wirelessly by means of short-range radio. This may enable an energy-saving data transmission. As indicated by the wavy lines between the modular ultrasonic devices 610-2 and 610-4 and the device 620 for evaluating measurement data in FIG. 6, the modular ultrasonic devices 610-2 and 610-4 may be configured accordingly to transmit the collected measurement data of the modular ultrasonic devices 610-1, . . . , 610-5 to the device 620 for evaluating measurement data by means of long-range radio.

In addition to the communication to a central instance, the modular ultrasonic devices 610-1, . . . , 610-5 may thus enable the formation of a sensor network and/or mesh 630 in order to transmit measurement data over longer distances despite the use of short-range radio. The insertion of individual modular ultrasonic devices and/or nodes with long-range radio functionality into the sensor network may help to reduce the susceptibility to errors (e.g. failure of a node would otherwise lead to an interruption of the communication).

The modular ultrasonic devices 610-1, . . . , 610-5 may alternatively also exchange data with one another in a wired manner. Likewise, the data exchange with the device 620 for evaluating measurement data may be carried out in a wired manner if required.

A communication channel may also be used for error communication in the local sensor network and/or mesh 630 to other modular ultrasonic devices. Thus, status messages may be transmitted in the sensor network 630. Also in normal operation, a measurement value communication may be carried out in the local sensor network and/or mesh 630 to other nodes and/or modular ultrasonic devices. Thus, measurement values or classifications such as, for example, “I-O” (“in order”) status or “N-I-O” (“not in order”) status of test objects may be transmitted. In the case of further modular ultrasonic devices in the sensor network and/or mesh 630, this may then trigger other and adapted measurements of the same test object in a test sequence, if applicable.

Furthermore, the modular ultrasonic devices 610-1, . . . , 610-5 may also be operated autonomously, for example, for long-term measurement tasks for condition monitoring (for example, in maintenance or in building monitoring, etc.) and collect data at fixed intervals. The data transfer may be carried out by means of the communication module, for example, according to a mobile radio standard.

Two or more of the modular ultrasonic devices 610-1, . . . , 610-5 may be set into a synchronized operation and, for example, one may operate only as a transmitter and one as a receiver.

If at least some of the modular ultrasonic devices 610-1, . . . , 610-5 are provided with a data memory according to the above explanations in the case of long-term measurement, it is possible to capture the stored data actively at specific time intervals (for example, when passing and/or walking by).

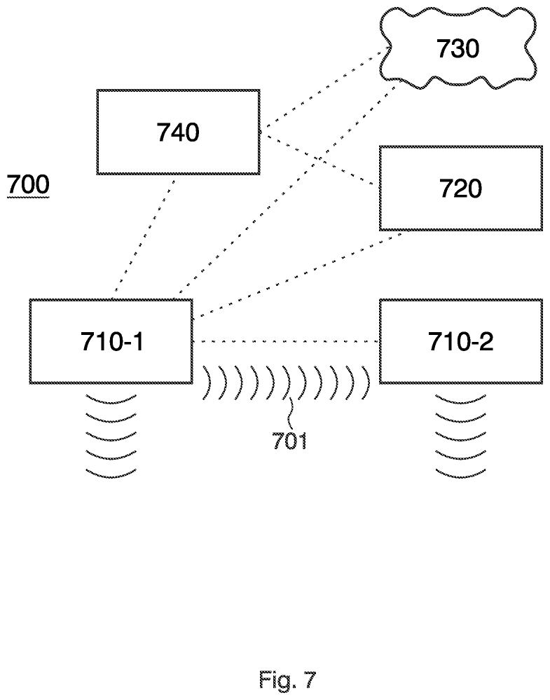

FIG. 7 shows a further ultrasonic system 700 having two modular ultrasonic devices 710-1 and 710-2. As indicated by the dashed line between the modular ultrasonic devices 710-1 and 710-2 in FIG. 7, the modular ultrasonic devices 710-1 and 710-2 are configured, analogously to the one described above, to exchange the measurement data wirelessly by means of short-range radio. In contrast to the ultrasonic system 600, the modular ultrasonic devices 710-1 and 710-2 are additionally configured to exchange the measurement data by encoding into the emitted ultrasonic waves 701. The emitted ultrasonic waves 701 are encoded according to the principles described above. Thus, a further communication channel is available to the modular ultrasonic devices 710-1 and 710-2.

In the example of FIG. 7, the modular ultrasonic device 710-1 is the predetermined ultrasonic device of the ultrasonic system 700 and forwards the collected measurement data of the modular ultrasonic devices 710-1 and 710-2 for evaluation. As indicated in FIG. 7, the predetermined ultrasonic device 710-1 may forward the collected measurement data not only to a device for evaluating measurement data, as is the case in the embodiment of FIG. 6, but also to multiple devices 720, 730 and 740 for evaluating measurement data.

The individual devices 720, 730 and 740 may be differently powerful and designed for different applications. For example, the device 720 may be an edge AI system that evaluates the collected measurement data of the modular ultrasonic devices 710-1 and 710-2 by means of AI-based edge computing. Similarly, the device 730 may, for example, be a cloud AI system that performs an AI-based evaluation of the collected measurement data of the modular ultrasonic devices 710-1 and 710-2 in the cloud. The device 740 may, for example, be a mobile terminal device such as a tablet computer or a mobile telephone on which software for the evaluation and/or visualization of the collected measurement data of the modular ultrasonic devices 710-1 and 710-2 is run. The devices 720, 730 and 740 may also exchange data among one another and thus, for example, distribute individual steps of the evaluation and/or visualization of the collected measurement data of the modular ultrasonic devices 710-1 and 710-2 to the individual devices.

By means of a corresponding software concept, the hardware concept described above and/or the distributed sensor networks may be made adaptable efficiently and for specific applications. In addition to the software/firmware on the respective modular ultrasonic devices themselves (e.g. hardware control, sound beam control in the transmission and reception path, plug-in-based filtering for application-specific AI signal processing and parameter extraction, imaging and analysis or communication with terminal devices), the software may, for example, support the communication between multiple individual systems and the communication with local edge AI and remote cloud AI. Thus, the individual elements may be individually developed, optimized and used for the different application scenarios in order to also enable an application-specific modularity of the software concept analogously to the hardware.

By means of the app-based software concept, new application areas for the same hardware may likewise be opened up by software updates and thus the flexibility of the proposed modular ultrasonic devices may be increased. By the complete relocation of signal processing and imaging into the software, hardware resources and thus costs may be saved.

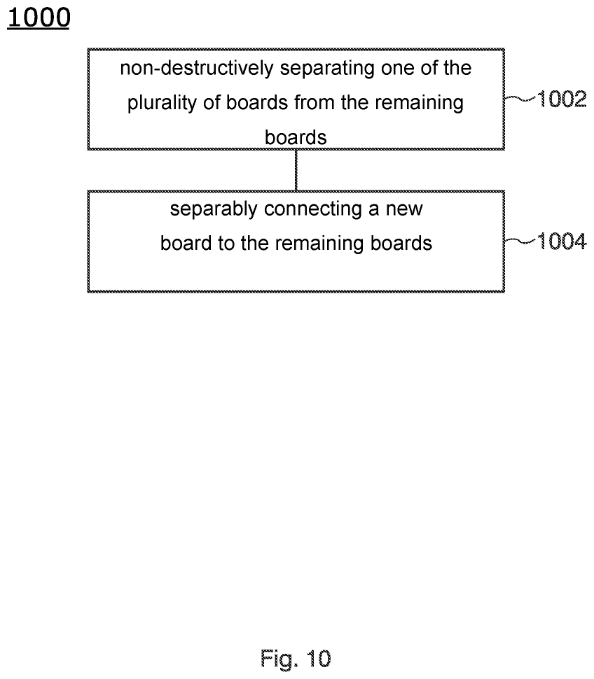

With regard to the later further development of the proposed ultrasonic system, the modular overall approach is of considerable advantage. On the one hand, the development time for new application fields and the associated “time-to-market” may be considerably reduced. On the other hand, the modular principle together with the variability of the software concept (e.g. downloadable apps) makes the system future-safe, flexible and cost-efficient in its further development. If a technological innovation leap is achieved in one of the individual modules (e.g. power supply, broadband radio standards), it is not necessary to redesign the entire system, but only to replace the corresponding module, as will be explained in more detail later with reference to FIG. 10.

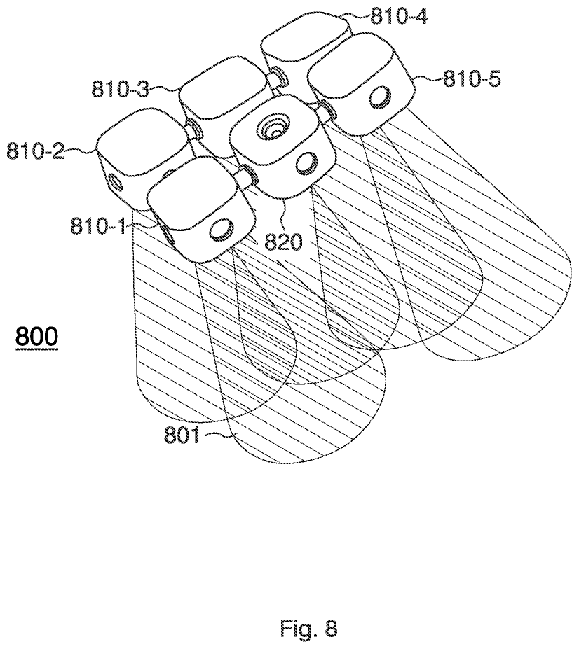

FIG. 8 shows a further ultrasonic system 800 having five modular ultrasonic devices 810-1, . . . , 810-5 according to the present disclosure. Analogously to the modular ultrasonic devices of the embodiments of FIGS. 6 and 7, the modular ultrasonic devices 810-1, . . . , 810-5 are each also configured at least according to the basic configuration described above and each additionally comprise the eighth board described above with the transceiver circuit. In the embodiment of FIG. 8, the housings of the five modular ultrasonic devices 810-1, . . . , 810-5 are fastened to one another via corresponding coupling elements in order to form a compact sensor network. In addition, an additional device 820 is coupled to the modular ultrasonic devices 810-1, . . . , 810-5. In the additional device 820, an accumulator is provided which provides the electrical energy for the power supply circuits of the modular ultrasonic devices 810-1, . . . , 810-5.

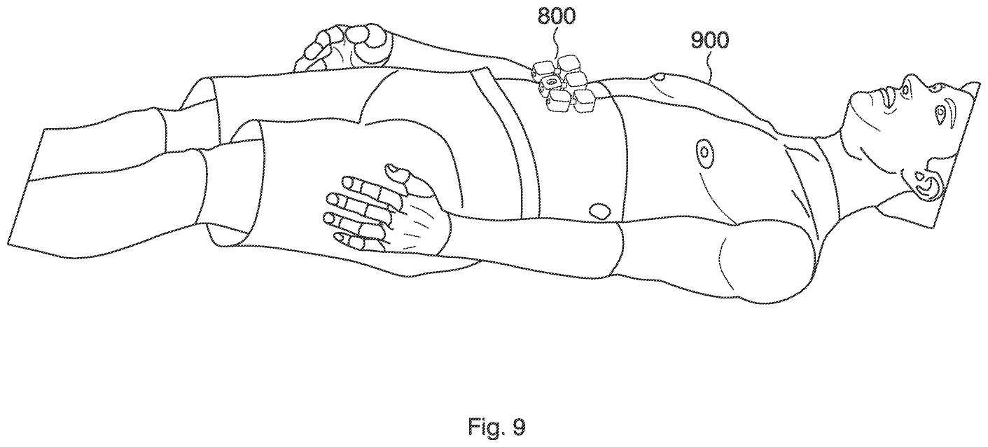

Via the hardware and software interfaces provided by the transceiver circuits of the modular ultrasonic devices 810-1, . . . , 810-5, a combination and networking of multiple modular ultrasonic devices via a wireless interface to a sensor network is possible. By means of the overlapping sound waves 801 of the modular ultrasonic devices 810-1, . . . , 810-5, a larger-surface area may be examined by means of ultrasound. Thus, by an integrated determination of the individual positions with respect to one another and by the software-based networking of the individual measurement data, the parallel and/or simultaneous acquisition of larger areas is possible by a separate device for evaluating measurement data, not illustrated in FIG. 8. This is illustrated by way of example in FIG. 9, where the ultrasonic system 800 is used for the ultrasound examination of the abdomen of a person 900.

As already indicated several times above, the modular ultrasonic devices according to the present disclosure enable the exchange of individual modules and/or boards. In order to illuminate this aspect of the present disclosure once again in more detail, a method 1000 for modifying a modular ultrasonic device according to the present disclosure will be described in more detail below with reference to FIG. 10. For example, the method 1000 may be used to exchange one of the plurality of boards 120 of the modular ultrasonic device 100 shown in FIGS. 1 and 2.

The method 1000 comprises non-destructively separating 1002 one of the plurality of boards from the remaining boards of the plurality of boards by detaching at least a part of the plug connectors. For example, for this purpose, the housing of the modular ultrasonic device may first be non-destructively opened and the plurality of boards or at least a part thereof may be removed from the housing. In order to exchange, for example, the first board 121 in the modular ultrasonic device 100 shown in FIGS. 1 and 2, the plurality of boards 120 may, for example, be removed from the housing 110 and the first board 121 may subsequently be non-destructively separated from the remaining boards of the plurality of boards 120 by detaching the plug connectors, which connect the first board 121 to the third board 123

Furthermore, the method 1000 comprises separably connecting 1004 a new board to the remaining boards of the plurality of boards by means of one or more plug connectors. The new board may, for example, have the same or a similar functionality as the one of the plurality of boards, which is and/or has been separated from the remaining boards of the plurality of boards by detaching at least a part of the plug connectors. With reference to the above example, for example, a new board having another ultrasonic transducer may be connected to the third board 123 via the plug connectors. If the modular ultrasonic device 100 is to be used, for example, first for a first use and subsequently for a second use with different requirements for the ultrasound used (for example, ultrasound geometry or ultrasound frequency), the modular ultrasonic device 100 may be adapted in a simple way to the respective use by exchanging the board having the at least one ultrasonic transducer.

Likewise, according to the method 1000, defective boards may, for example, be exchanged in a simple way. Since only the respective board has to be exchanged in the event of a defect, a modular ultrasonic device according to the present disclosure may be repaired in a simple and cost-effective way. Boards for certain functionalities may also be updated according to the progress of the technology. For example, in the event of the emergence of a new communication standard, the board having the transceiver circuit according to the method 1000 may be exchanged in a simple way for a more modern board having a transceiver circuit which supports the new communication standard, so that a modular ultrasonic device according to the present disclosure may also communicate with the most modern third-party devices.

According to the present disclosure, a kit for ultrasonic applications having a modular hardware base and various software apps (for example, in the system, in the mobile device, in the local edge AI system, in the cloud) is provided. The kit enables the addressing of a very wide application area by application-specific combination. If it is not sufficient or if large technological innovation leaps occur (for example, new battery technology or radio technology), only one sub-module and/or one board has to be redeveloped, so that the modular ultrasonic device may be adapted in a very simple way by exchanging the corresponding module and/or the corresponding board and, depending on the use, by adapting software, if applicable.

Advantages of the kit are therefore a wide range of applications by simple reconfiguration and also great future security, since new technologies may be easily transferred into corresponding modules and/or boards. Thus, the concept is also very sustainable (new applications and technologies do not always require complete redevelopment and production) and development costs are reduced to a great extent. A user may always use the current state of the art in all sub-modules and/or boards in the long term and may technologically extend the system by further sensor classes if required (new modules for, for example: pressure measurement, brightness, smell, humidity, hardness, etc.). Future technology leaps in individual components may be directly utilized by exchanging the corresponding module, without having to carry out a redesign of the entire electronics unit.

With the proposed kit, it is possible to develop new ultrasonic systems and methods in a perspectively simpler, faster, more sustainable and more cost-effective manner. In addition, these may be flexibly adapted to a very wide range of possible different application fields (e.g. care, fitness, Industry 4.0. medicine, etc.) by updates of the software modules and different apps with the same hardware basis for the measurement technology.

In the case of a technologically wide configuration of the hardware, a very wide range of applications is covered solely by the application-specific software modules. If the performance of an individual hardware module and/or an individual board is not sufficient for an application, the individual modules and/or boards may be selected and freely recombined very simply by the kit approach with regard to the respective application.

The high flexibility and cost efficiency of the technology approach results in a very wide range of applications. For example, the application is possible in the medical and/or care sector, in the consumer and/or prosumer sector or in the industrial sector.

The present disclosure indicates modular ultrasonic sensor electronics and software, which may be flexibly configured for various application fields, and is so cost-effective that mass markets may be addressed in the medical as well as technical context.

The plurality of different modules and/or boards described above may be combined into an overall system depending on the application. Requirements with regard to the application-specific frequency range, the output power, etc., define the number of necessary modules and/or boards, which are flexibly configured via, for example, standardized hardware and software interfaces. Accumulator-based solutions are just as conceivable as are also memory modules for recording the data of long-term measurements. Multiple of these ultrasonic systems may also be combined into an intelligent sensor network via a wireless interface, for example, and resources may be shared between the sensor systems. For the communication and for the data transfer between a mobile terminal device and the modular ultrasonic device, a module having a manufacturer-independent radio interface or a cable connection having a standardized communication protocol may be integrated, for example. Thus, the system may be simply connected to each terminal device of corresponding power and started after loading the corresponding application-related software.

The present disclosure indicates an autonomously operating overall concept, which is very cost-effective and, in addition, depending on the need and application, either integrates the steadily increasing performance of mobile consumer terminal devices via radio interfaces (e.g. Bluetooth, WLAN, etc.) and thereby uses the “intelligence” from the hardware to the software running on these consumer devices, or uses these mobile terminal devices as a bridge to AI cloud applications, for example, or has a connection to the cloud directly via the integrated hardware and enables AI applications there. Automated intelligent evaluation methods may thus be carried out in a cloud network or as edge AI having integrated data processing capacity.

The aspects and features described in relation to a particular one of the previous examples may also be combined with one or more of the further examples to replace an identical or similar feature of that further example or to additionally introduce the features into the further example.

It is further understood that the disclosure of several steps, processes, operations or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process, or operation may include and/or be broken up into several sub-steps,-functions,-processes or-operations.

If some aspects in the previous sections have been described in relation to an apparatus or system, these aspects should also be understood as a description of the corresponding method. In this case, for example, a block, an apparatus or a functional aspect of the device or system may correspond to a feature, such as a method step, of the corresponding method.

Accordingly, aspects described in relation to a method shall also be understood as a description of a corresponding block, a corresponding element, a property or a functional feature of a corresponding device or a corresponding system.

The following claims are hereby incorporated in the detailed description, wherein each claim may stand on its own as a separate example. It should also be noted that—although in the claims a dependent claim refers to a particular combination with one or more other claims—other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

1. An ultrasonic system comprising:
a plurality of modular ultrasonic devices; and
a device for evaluating measurement data,
wherein the plurality of modular ultrasonic devices are configured to transmit respective measurement data directly or by forwarding via at least one further of the plurality of modular ultrasonic devices to one or more predetermined modular ultrasonic devices of the plurality of modular ultrasonic devices, and
wherein the one or more predetermined modular ultrasonic devices are configured to transmit the collected measurement data of the plurality of modular ultrasonic devices to the device for evaluating measurement data.
wherein the device for evaluating measurement data is configured to determine one or more predetermined characteristics based on the measurement data, and
wherein the plurality of modular ultrasonic devices each comprise:
a housing;
at least one ultrasonic transducer configured to:
based on a respective control signal, generate and emit ultrasonic waves; and
depending on received ultrasonic waves, generate a respective measurement signal; and
a plurality of boards arranged in the housing, which are separably connected to one another via respective plug connectors, wherein the plurality of boards comprise at least the following:
a first board with a power supply circuit configured to generate a respective power supply signal for the at least one ultrasonic transducer and the further boards of the plurality of boards;
a transmit circuit configured to generate the respective control signals for the at least one ultrasonic transducer; and
a receive circuit configured to process the respective measurement signals of the at least one ultrasonic transducer,
wherein the transmit circuit and the receive circuit are either both formed on a second board of the plurality of boards or the transmit circuit is formed on the second board and the receive circuit is formed on a third board of the plurality of boards,
wherein the at least one ultrasonic transducer is formed on a fourth board of the plurality of boards or is separably connected to one of the plurality of boards, and
wherein the plurality of boards further comprise a fifth board with a transceiver circuit configured to:
based on data of the mobile ultrasonic device to be transmitted, generate a transmit signal;
based on a receive signal, determine receive data for the mobile ultrasonic device;
apply the transmit signal to an antenna of the modular ultrasonic device for emission into surroundings of the modular ultrasonic device and/or apply the transmit signal to an interface of the mobile ultrasonic device for wired communication with an external device and/or forward the transmit signal to the transmit circuit in order to generate the respective control signal for the at least one ultrasonic transducer based on the transmit signal and thus to encode the data of the mobile ultrasonic device to be transmitted into the ultrasonic waves emitted by the at least one ultrasonic transducer; and
receive the receive signal from the antenna and/or receive the receive signal from the interface for wired communication with the external device and/or receive the receive signal from the receive circuit, wherein the receive circuit is configured to derive the receive signal from the respective measurement signal of the at least one ultrasonic transducer.

2. The ultrasonic system according to claim 1, wherein the plurality of boards are arranged one above the other and successive boards of the plurality of boards are respectively plugged together by means of the plug connectors.

3. The ultrasonic system according to claim 1, wherein the plurality of boards are arranged next to one another.

4. The ultrasonic system according to claim 1, wherein the housing comprises a window with increased acoustic transparency compared to the rest of the housing, and wherein the window is arranged in front of the at least one ultrasonic transducer.

5. The ultrasonic system according to claim 1, wherein the plurality of boards further comprise:
a sixth board with a processing circuit, which is configured to recognize a respective functionality of the plurality of boards and, based thereon, to control a data exchange between at least a part of the plurality of boards.

6. The ultrasonic system according to claim 5, wherein the processing circuit is further configured to determine one or more predetermined characteristics based on the respective measurement signal of the at least one ultrasonic transducer processed by the receive circuit.

7. The ultrasonic system according to claim 6, wherein the processing circuit is further configured to determine the one or more predetermined characteristics using a model trained by machine learning.

8. The ultrasonic system according to claim 1, wherein the plurality of boards further comprise:
a seventh board having a data memory configured to store the respective measurement signal of the at least one ultrasonic transducer after processing by the receive circuit.

9. The ultrasonic system according to claim 1, wherein the plurality of boards further comprise:
an eighth board with a position sensing circuit configured to determine an absolute position of the modular ultrasonic device and/or a relative position of the modular ultrasonic device with respect to a predetermined object.

10. The ultrasonic system according to claim 1, wherein the receive circuit is configured to at least amplify and digitize the respective measurement signal of the at least one ultrasonic transducer during processing.

11. The ultrasonic system according to claim 1, wherein the second board further comprises an accumulator coupled to the power supply circuit, and wherein the power supply circuit is configured to generate the respective power supply signal based on energy stored in the accumulator.

12. The ultrasonic system according to claim 1, wherein a socket for connection to a charging cable is integrated into the housing, and wherein the power supply circuit is configured to generate the respective power supply signal based on electrical energy received at the socket.

13. The ultrasonic system according to claim 1, wherein a fastening element for fastening the modular ultrasonic device to a test object is integrated into the housing.

14. The ultrasonic system according to claim 1, wherein the housing extends in three spatial directions perpendicular to one another and an extension of the housing in each of the three spatial directions perpendicular to one another is less than 15 cm.

15. The ultrasonic system according to claim 1, wherein the plurality of modular ultrasonic devices each further comprise an energy converter configured to convert ambient energy from the surroundings of the modular ultrasonic device into electrical energy, and wherein the power supply circuit is configured to generate the respective power supply signal based on the electrical energy provided by the energy converter.

16. The ultrasonic system according to claim 1, wherein the plurality of plug connectors are configured to conduct the respective power supply signal from the second board to the respective further board of the plurality of boards.

17. The ultrasonic system according to claim 1, wherein the plurality of plug connectors comprise signal paths for the exchange of data between at least a part of the plurality of boards.

18. The ultrasonic system according to claim 1, wherein the plurality of modular ultrasonic devices each further comprises a user interface integrated into the housing, which is configured to:
optically and/or acoustically output information relating to the modular ultrasonic device to a user; and/or
receive a user input of the user.

19. (canceled)

20. (canceled)

21. The ultrasonic system according to claim 1, wherein the plurality of modular ultrasonic devices are configured to transmit the measurement data to the one or more predetermined modular ultrasonic devices of the plurality of modular ultrasonic devices wirelessly by means of short-range radio and/or encoded into the respectively emitted ultrasonic waves.

22. The ultrasonic system according to claim 1, wherein the device for evaluating measurement data is configured to determine the one or more predetermined characteristics from the measurement data using a model trained by machine learning.

23. A method for modifying one of the modular ultrasonic devices in the ultrasonic system according to claim 1, the method comprising:
non-destructively separating one of the plurality of boards from the remaining boards of the plurality of boards by detaching at least a part of the plug connectors; and
separably connecting a new board to the remaining boards of the plurality of boards by means of one or more plug connectors.

24. The method according to claim 23, wherein the new board has the same functionality as the one of the plurality of boards which is separated from the remaining boards of the plurality of boards by detaching at least a part of the plug connectors.
